(19) United States
(12) Patent Application Publication
(10) Pub. No.: US 2013/0324810 A1
(43) Pub. Date: Dec. 5, 2013

(54) Cerebral and Retinal Perfusion Monitoring Systems and Devices

(71) Applicant: Yuri GELLAND, San Diego, CA (US)

(72) Inventor: Yuri GELLAND, San Diego, CA (US)

(21) Appl. No.: 13/907,916

(22) Filed: Jun. 1, 2013

Related U.S. Application Data
(60) Provisional application No. 61/654,739, filed on Jun. 1, 2012.

(51) Int. Cl. A61B 5/026; A61B 3/14; A61B 3/12; A61B 17/02

(52) U.S. Cl. CPC A61B 5/026; A61B 17/02; A61B 3/145; A61B 3/1241
USPC 600/323; 600/236; 351/206; 600/398; 600/407

(57) ABSTRACT

Cerebral and retinal perfusion monitoring devices, systems, and methods, are described. Such devices, systems, and methods can be used during surgical procedures, including cardiac surgery with cardiopulmonary bypass and spine surgery in prone position, to monitor a patient's brain perfusion in real time. In some embodiments, such monitoring allows trends in perfusion over time to be assessed. Such monitoring allows appropriate medical interventions to be undertaken in the event a state of hypoperfusion is detected, thereby reducing risks of brain hypoperfusion and stroke for patients undergoing heart surgery with cardiopulmonary bypass, as well as minimizing risk of retinal artery thrombosis with resulting blindness for patients undergoing prolonged procedures in the prone position.

### RELATED APPLICATION

This application claims the benefit of and priority to co-pending U.S. provisional patent application Ser. No. 61/654,739, filed 1 Jun. 2012, the contents of which is hereby incorporated by reference in its entirety for any and all purposes.

### TECHNICAL FIELD

The present invention relates generally to monitoring blood perfusion and other conditions of a human patient.

### BACKGROUND

Stroke, perioperative visual loss, and postoperative cognitive dysfunction remain among the most serious perioperative complications, affecting hundreds of thousands of patients in the United States and worldwide. Stroke and visual loss, although relatively rare, usually carry catastrophic consequences for the patient, resulting in death or prolonged disability. The associated annual loss of productivity and monetary costs to health care providers, patients, and the society is substantial.

In addition to stroke and visual loss, another perioperative complication—postoperative cognitive dysfunction, affects a great number of patients every year. All these serious events are more common after major surgical procedures, such as cardiac surgery with cardiopulmonary bypass, vascular surgery, neurosurgery, and major back surgery.

The etiology of stroke is multifactorial, including thromboembolic events, ischemic strokes, and hemorrhagic strokes. In most instances, intraoperative stroke is detected only long after the fact, when the patient fails to wake up or wakes up postoperatively with neuromotor deficit.

Intraoperative visual loss is a result of retinal artery thrombosis or prolonged hypoperfusion of a patient's retina.

During major surgery, including cardiac surgery and neurosurgery, a patient's physiologic parameters change rapidly and continuously, and can result in periods of insufficient blood flow and oxygen delivery to the brain. Without a dedicated monitor for cerebral blood flow, some of these intraoperative events remain undetected by operating room personnel and can result in perioperative complications. Furthermore, as these periods are usually brief and transient, it is very difficult to establish cause—effect relationships in retrospective analyses. The fact that the irreversible anoxic damage to the brain cortex begins within minutes (typically less than 5 min.) of insufficient blood flow, demonstrates the importance of real time intraoperative monitoring of cerebral perfusion.

Currently there are no adequate monitoring devices available for use in operating rooms and intensive care units to evaluate cerebral perfusion in real time. Therefore, proxies or substitute parameters are used to extrapolate physiologic data in order to assess the status of the brain's well being. Commonly, blood pressure monitoring, pulse oximetry, EKG, and respiratory gas analysis, are routinely used perioperatively for this purpose. In addition, intracranial pressure can be monitored when indicated during neurosurgery and postoperatively. Near infrared pulse oximetry has recently been introduced for real time non-invasive measurement of oxygen content in the brain.

Although the information on cerebral and retinal blood flow is absolutely vital for assessment of well being of the brain and eyes, to date no system has been developed for this purpose. This invention addresses this long standing but unsatisfied need using something that has always been available: the relatively accessible and vital “window to the brain”—the fundus of a patient's eyes.

### SUMMARY OF THE INVENTION

This invention concerns intraoperative patient monitoring systems that allow real time monitoring of a patient's brain perfusion, and, if desired, monitoring of additional parameters such as intraocular pressure and retinal perfusion, during surgical procedures including, for example, cardiac surgery with cardiopulmonary bypass and spine surgery with the patient in the prone position. Use of these patient monitoring systems will reduce the risks of brain hypoperfusion and stroke, as well as reduce the risk of retinal artery thrombosis, which can result in blindness for patients undergoing prolonged surgical procedures in the prone position.

Thus, in one aspect, the invention concerns patient monitoring systems that include an imaging system and one or more, preferably a pair of, corneal caps. The imagining system is configured to image retinal vasculature in the eye through the corneal cap on which the corneal cap is positioned. Generally, such an imagining system comprises an image sensor for each eye to be monitored, a light source associated with each image sensor and positioned to illuminate the retina of the eye to be monitored, a mount configured to position the video camera(s) and light source(s) in front of a patient's eyes, and, optionally, a power supply. In preferred embodiments the image sensor is a video camera, preferably a high definition video camera. The light source can be any suitable light source. Light sources based on one more LEDs are preferred.

An imaging system mount is any structure configured to allow easy positioning of the imaging system on and removal from a patient's head. The mount positions the image sensor(s) and associated light source(s) to allow imaging of the retinal vasculature of the patient's eye(s) to be monitored. Representative mounts are those based on goggle or safety glasses, which are adapted to carry and properly position the imaging system carried thereon.

A corneal cap is typically a sterily packaged single use, disposable article that comprises a contact lens portion having an eyelid retractor portion integrated on the outer surface of the contact lens portion. The contact lens portion is preferably transparent, but in some embodiments can be opaque or translucent. A corneal cap can be made from any suitable biocompatible material(s) using any suitable manufacturing method.

In some embodiments, a patient monitoring system of the invention also includes one or more other sensors, for example, a pulse oximeter and/or an intraocular pressure sensor positioned to obtaining readings the eye being monitored.

A related aspect concerns kits that comprise one, two, or more corneal caps and imaging systems according to the invention.

Another aspect of the invention relates to methods of monitoring blood flow or perfusion in a patient. Such methods are accomplished by positioning a patient monitoring system according to the invention on a patient and monitoring blood flow in the retinal vasculature of at least one eye of the patient.

Still another aspect of the invention concerns automated methods of continuous monitoring of retinal blood flow to assess in real time cerebral blood perfusion. Such methods involve using a patient monitoring system of the invention to monitor blood flow in the retinal vasculature of at least one eye of the patient in conjunction with using a computer to process image data from the imaging system to generate a measured or derived index number reflecting a parameter associated with retinal blood flow. The index number can be output to a user, wherein a change in the index number reflects a change in the parameter(s) being monitored. In some embodiments, the index number is indicative of a diameter of a retinal blood vessel, a number of pixels, optionally red pixels, in a selected area in one or more images captured by the imaging system, or a percentage change from a baseline.

Another aspect involves methods of monitoring bilateral retinas in real time. These methods use a patient monitoring system of the invention to generate image data that allows for real time comparison of retinal blood flow in a patient's left eye versus the patient's right eye.

These and other features and aspects of the present invention will be better understood with reference to the following description, drawings, and appended claims.

### Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

In this specification, the words “embodiment” and “variant” refer to particular apparatus, process, or article of manufacture, and not necessarily to the same apparatus, process, or article of manufacture. Thus, “one embodiment” (or a similar expression) used in one place or context can refer to a particular apparatus, process, or article of manufacture; the same or a similar expression in a different place can refer to a different apparatus, process, or article of manufacture. The expression “alternative embodiment” and similar expressions and phrases are used to indicate one of a number of different possible embodiments. The number of possible embodiments is not necessarily limited to two or any other quantity. Characterization of an item as “exemplary” means that the item is used as an example. Such characterization of an embodiment does not necessarily mean that the embodiment is a preferred embodiment; the embodiment may but need not be a currently preferred embodiments. All embodiments are described for illustration purposes and are not strictly limiting.

The words “couple,” “connect,” and similar expressions with their inflectional morphemes do not necessarily import an immediate or direct connection, but include connections through mediate elements within their meaning.

A “patentable” process, machine, system, device, or article of manufacture according to the invention means that the invention as claimed satisfies all statutory requirements for patentability at the time the particular analysis is performed. For example, with regard to novelty, non-obviousness, or the like, if post-issuance investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards for assessing whether a particular statutory requirement for patentability is satisfied change from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

### BRIEF DESCRIPTION OF THE DRAWINGS

A brief description of the drawings is provided below.

FIG. 1 is a cross-sectional illustration of some of the major arterial branches of the common carotid artery in the head of a patient. Arteries are labeled.

FIG. 2 is a drawing that also shows the some of the major arteries in the head of a patient, including the ophthalmic artery that feeds the retinal artery (not shown). Some of the arteries are labeled.

FIG. 3 is a cutaway diagram of the human eye, with some of the major features being labeled. Retinal vasculature is also shown, as is light, which enters through the pupil.

FIG. 4 is a drawing that illustrates some of the vasculature visible in the retina, including branches of the retinal artery and retinal vein.

FIG. 5 is a drawing that shows two corneal caps of the invention. The contact lens and eyelid retractor portions are labeled.

FIG. 6 is a cross-section of a representative corneal cap of the invention positioned on a patient's cornea.

FIG. 7 is an illustration of a patient monitoring system according to the invention, including a corneal cap and an imaging system.

FIG. 8 is an illustration that shows a patient monitoring system of the invention positioned on a simulated patient.

### DETAILED DESCRIPTION OF THE INVENTION

This invention provides a new, patentable patient monitoring system for the operating room and intensive care units that addresses the unmet demand for continuous monitoring of a patient's cerebral and retinal blood flow. The purpose of such monitoring is to reduce the incidence of serious perioperative complications such as ischemic strokes, intraoperative visual loss, and perioperative cognitive dysfunction.

The devices of the invention are novel, non-invasive, real-time patient monitoring systems configured for routine monitoring of patients undergoing surgery, including cardiac surgery, neurosurgery, major vascular surgery, and orthopedic surgery. The inventive monitors can be used in operating rooms and surgical suites around the world. As described herein, the instant monitoring modalities employ a re-useable imaging system and single use, disposable components termed “corneal caps”. As presently envisioned, in use a new set of corneal caps will be required for each surgical procedure. A detailed description of the invention, including representative, non-limiting examples, is provided below.

Anatomy

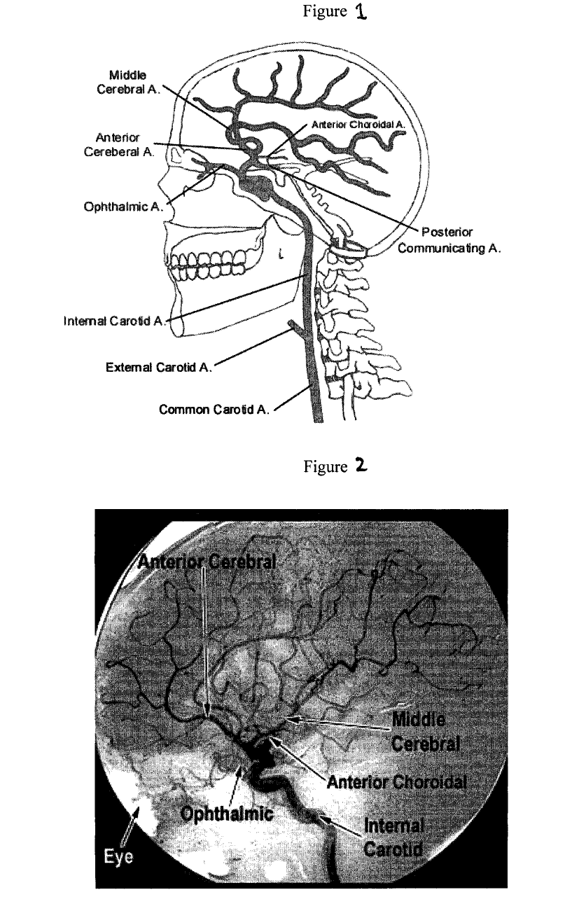

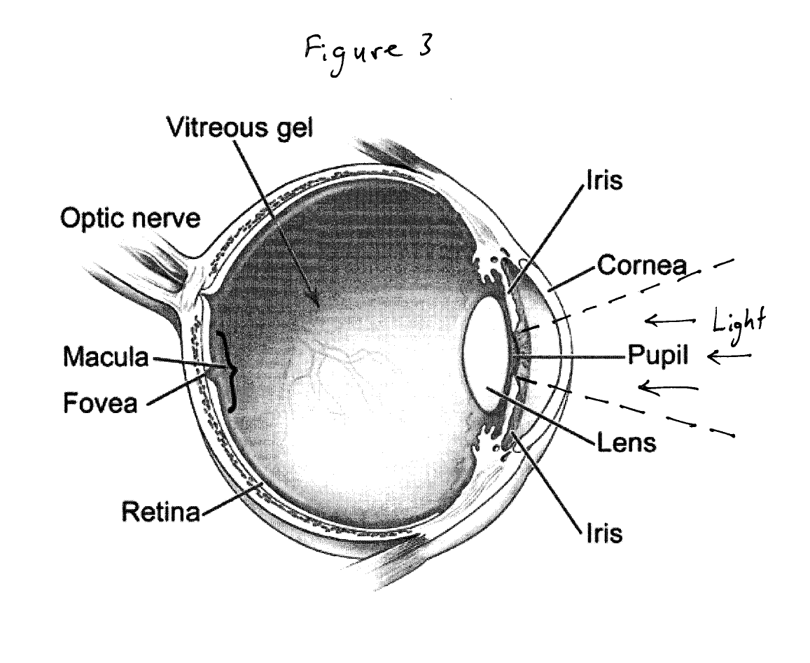

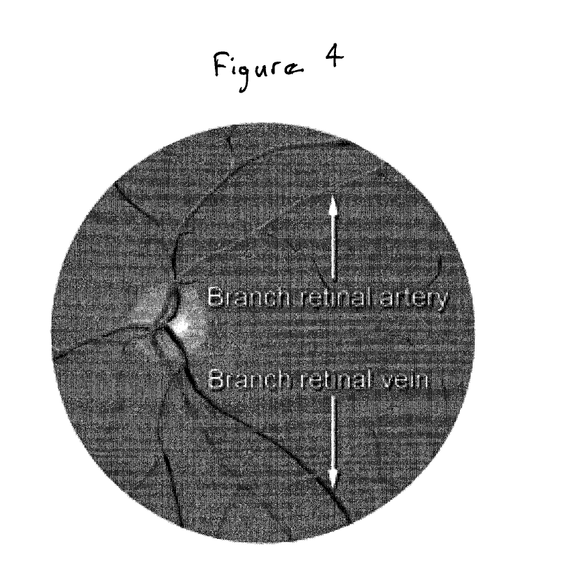

Arterial blood flow to the brain and eyes is supplied mostly by the left and right internal carotid arteries and vertebral arteries (see FIG. 1). The ophthalmic artery is a first branch from the internal carotid artery and in turn supplies the retinal artery (see FIGS. 1 and 2). The retinal artery, retinal vein, and ophthalmic nerve can easily be observed and non-invasively monitored intraoperatively (see FIGS. 3 and 4). As is well known to retina surgeons, the status of blood flow in the retinal blood vessels (see FIG. 4) can be directly observed under the operating microscope during surgical procedures on the retina; however, such observation is outside of everyday practice for anesthesiologists, neurologists, neurosurgeons, cardiologists, and vascular surgeons. Indeed, an anesthesiologist monitoring a patient's systemic blood pressure using conventional blood pressure monitoring technology would have no way to determine if the flow of blood through a patient's retinal or other intracranial artery(ies) had been reduced or completely ceased due, for example, to a low systemic blood pressure, blood clot, or hemorrhage in one of the blood vessels in a patient's eye or head.

Using a patient monitoring system according to the invention, however, retinal blood perfusion can be observed and measured, providing valuable insight about the status of cerebral perfusion, as well. If, for example, retinal perfusion is determined by the system to be inadequate while the patient's systemic blood pressure remains within low-normal ranges, this indicates that the parts of brain that receive blood supply from the same internal carotid artery may be also at risk for hypoperfusion, which could lead to cognitive dysfunction, ischemic stroke, or visual loss if appropriate treatment is not promptly delivered.

System Components

The patient monitoring systems of the invention provide noninvasively obtained, real time video images (preferably high definition video images) of the retinal blood vessels of one, and preferably both, of a patient's eyes. The video images can be displayed on operating room monitors in real time, giving doctors and other operating room personnel an opportunity to immediately address intracranial blood perfusion problems as they occur, thereby reducing the risk of postoperative complications attributable to intracranial hypoperfusion. Alternatively or concurrently, video image data can be processed and compared to a baseline using a computer to determine if the patient's retinal perfusion is within normal or abnormal ranges. Results of the computer-based analysis can then be displayed or otherwise output to operating room personnel (e.g., the anesthesiologist) on a continuous or periodic basis.

In general, a patient monitoring system according to the invention will include the following components: a transparent (clear) corneal cap for each of a patient's eyes and an imaging system that allows imaging of the retinal vasculature of eye(s) to be monitored.

A corneal cap is a single use, disposable contact lens-eyelid retractor that can be positioned on the cornea of a patient's eye. A corneal cap allows light to enter a patient's eye so that the retinal vasculature can be observed.

A corneal cap according to the invention includes a contact lens portion and an eyelid retractor. The contact lens portion is a thin, transparent (or translucent), plastic lens placed directly onto the surface of the cornea of a patient's eye. In some embodiments, the contact lens portion covers more than the patient's cornea, up to and including most if not substantially all of the sclera visible to an observer of the patient's opened eye.

A corneal cap can be manufactured from any suitable biocompatible material, or combination of materials, now known or later developed using any suitable process. Preferred materials for rigid corneal caps include polymethyl methacrylate (PMMA), as well as gas-permeable materials suitable for making rigid contact lens. Here, “rigid” means that material is a slightly flexible plastic or polymer, and is preferably a material that is gas-permeable so as to allow oxygen (and other gases) to pass through the material to the eye on which the lens (i.e., corneal cap) has been placed. Preferred materials for corneal caps wherein the contact lens portion is soft include gas-permeable polymeric hydrogels, particularly silicone-based hydrogels used for making corrective contact lenses. Corneal caps wherein the contact lens portion is a hybrid of rigid and soft materials are also envisioned. As will be appreciated, because a corneal cap is intended to prevent eyelid closure, the materials from which the cap is constructed will have sufficient mechanical integrity so as to prevent collapse or substantial movement during a blink reflex.

Corneal caps can be made by any suitable manufacturing method, including molding (e.g., injection molding), casting (e.g., spin casting), and machining (e.g., by diamond turning on a CNC lathe). The component parts of a corneal cap, the contact lens portion and eyelid retractor portion(s), can be manufactured separately and then be combined. Alternatively, they can be made together, preferably as a single, integrated part.

The eyelid retractor portion of a corneal cap protrudes from the cup's outer surface a distance sufficient to prevent eyelid closure over a corneal cap when positioned on the cornea of a patient's eye. Suitable distances include from 0.1 millimeter (mm) to about 12 mm, preferably about 0.5 mm to about 5 mm. The retractor portion can comprise a continuous protruding element. Alternatively, the retractor portion can be comprised of one or more spaced subunits. The retractor portion can be inwardly or outwardly tapered, or not tapered.

Two corneal caps will typically be used for each patient, one on each eye. The corneal caps will be placed directly on patient's corneas (or corneal surfaces), providing protection from abrasions, dryness, and compensation for optical aberration of human eye. A corneal cap preferably conforms to its respective cornea and remains in close contact with the cornea throughout the surgical procedure during which monitoring occurs. Each corneal cap includes a contact lens portion, which contacts the cornea, and eyelid retractors that keep the eyelids in the open position even in an anesthetized patient. The corneal caps can be made from any suitable material, or combination of materials. After manufacture, they are preferably packaged and sterilized.

In some embodiments, a corneal cap may also include a light source, for example, one or more LEDs, and a power supply, typically one or more batteries to power the light source. The light source is exposed on the inner surface of the corneal cap such that when positioned on a cornea, the cap can illuminate the eye's retina. In such embodiments, it is not necessary for the corresponding imaging system to include a separate light source associated with the image sensor, although systems that include an image sensor-associated light source are also envisioned.

The imaging system of the invention includes an image sensor, typically a video camera, preferably a high definition video camera, to monitor the retinal vasculature of each eye upon which a corneal cap has been placed, a light source to illuminate the retina being imaged, and a mount for securely positioning the video camera(s) and light source(s) in front of a patient's eyes in order to allow for imaging of the patient's retinal blood vasculature, and a power supply for the video camera(s) and light source(s). In some embodiments, the power supply is provided separately, and is not part of the imaging system itself but is connected to the imaging system prior to use in order to provide the necessary electrical energy to the imaging system electronics.

Any suitable video camera can be adapted for use in the present invention. Preferred video cameras are small, lightweight high definition video cameras such as are used in smart phones. Here, “high definition” refers to high definition video, as used in HD television (HDTV) broadcasting, which is generally understood to refer to video images having more than 480 horizontal lines of resolution, preferably 720 lines, and even more preferably, 1080 or more horizontal lines, with interlaced (i) or, preferably, progressive (p) scanning, preferably at frame rates of at least about 24 or 25 frames per second, preferably 30-60 or more (120, 1,000, 1,200, 1,800, 2,400, 2,700) frames per second or more. A typical 720p video mode produces a frame size of 1,280×720 pixels (W×H), while 1080i and 1080p video produce a frame size of 1,920×1,080 pixels (W×H). Ultra HC video modes of 2000, 2160p, 2540p, 4000p, and 4320p are also known and may be adapted for use in the practice of this invention. Image data from such cameras can be transmitted to video monitors in any suitable fashion, including wirelessly (in which event an appropriate transmitter and/or transceiver will also be included) or via suitable wiring or cabling.

Any suitable light source can be adapted for use in an imaging system of the invention. Preferred light sources include one or more LEDs. In general, each video camera will have an associated light source. Preferably a light source will illuminate at least a portion of the retina to be imaged by the video camera with which it is associated. The area of a retina to be imaged will depend on many factors, including the field of view of the lens of the video camera being used, the distance between the video camera lens and the patient's eye, etc.

Any suitable power supply can be adapted to power the electronics of the particular imaging system. Suitable power supplies include those that employ one or more batteries to provide electricity, as well as those intended for connection to a hard-wired electrical system in the operating room.

Any suitable mount for the imaging system of the invention can be employed. A representative example of such a mount is a pair of goggles configured to securely carry the imaging system of the invention and position its video camera(s) for sustained imaging of a patient's retinal vasculature, as shown, for example in FIG. 4. As will be appreciated, a mount such as a pair of goggles or safety glasses adapted for use in the invention can also provide additional protection for a patient's eyes while they are open in the operating room. Mounts such as goggles or safety glasses will also preferably provide an air gap between the corneal caps and the interior surface of the goggles or glasses (or other shielding device) in order to minimize the risk of eye injury.

Optional components for the imaging system include one or more monitors for displaying video images from the system's video camera(s) so that real time video, and, if desired, still images, of a patient's left and right retinal blood vessels and optic discs can be viewed by operating room personnel. Other optional components include a pulse oximeter and/or in intraocular pressure sensor. For example, a pulse oximeter can provide real time readings of oxygen content of the patient's retinal blood, which data the system can output to a monitor or other information display device in any desired format. The pulse oximeter can be any suitable pulse oximetry device, preferably a reflectance pulse oximeter that allows continuous monitoring of retina arterial oxygen saturation. Similarly, an intraocular pressure sensor can provide continuous or continual real time measurements of intraocular pressure, which data the system can output to a monitor or other information display device in any desired format.

The components of the imaging system may be connected to a remote monitoring system using one or more electrical, optical, and/or wireless connections.

The data from all or some of the sensors included in a system of the invention may be provided to the same physical display (or other output or storage device) to facilitate monitoring by medical personnel, who may include an anesthesiologist monitoring the patient during a surgical procedure. The video image of the retinal blood vessels, which, if desired, may also be output with data regarding parameters such as retinal pulse oximetry and/or intraocular pressure (provided such optional sensors are included with the particular patient monitoring system), can be continuously monitored in real time by, for example, an anesthesiologist for signs of ischemia and inadequate intracranial and/or retinal perfusion.

As will be appreciated, data generated by a patient monitoring system of the invention may also (or instead) be recorded into any suitable mass data storage device(s) for future reference.

In some embodiments, image data from the video cameras can be processed for the purpose of non-invasively monitoring one or more parameters of blood flow in a patient's retinal vasculature. Such parameters include blood pressure, flow rate, level of oxygenation, blood vessel diameter, and the like. In some embodiments, an index number for trend monitoring of the status of blood flow in retinal arteries can be generated. In some of these embodiments, an index number will be continuously displayed and trended to standardize and simplify the analysis of blood flow. In order to accomplish this, an algorithm may be executed by one or more processors (of the external monitoring system or another computer-based device) to process various data (e.g., image, pressure, pulse oximetry, etc.) to generate one or more indicators of retinal and brain perfusion. The algorithm may also generate one or more automated alarms in response to the data meeting one or more predetermined criteria.

Such automated methods of continuous monitoring of retinal blood flow can be used for real time assessment of cerebral blood perfusion over time by generating a measured or derived index number, such as may be derived from the change in diameter of one or more retinal blood vessels (e.g., retinal artery, retinal vein) over time, the change over time in the absolute number (or a computer-derived derivative thereof) of red pixels in one or more selected areas (e.g., in the area that defines a portion of a retinal blood vessel) of one or more frames of video image data, the percentage change from the baseline, or from another observable metric within limits established by a user of the patient monitoring system. If desired, the system can be configured to trigger an alarm to alert attending medical personnel of a potential for brain hypoperfusion if the index or other number falls below pre-determined limit.

Applications

The patient monitoring systems of the invention allow an anesthesiologist and/or other operating room personnel to observe the status of a patient's retinal blood vessels in real time in order to assess cerebral and/or retinal blood flow during surgical procedures, including cardiac surgery with cardiopulmonary bypass and spine surgery in the prone position. If desired, trends, or changes over time, in blood flow (or another parameter associated with perfusion adequacy) can be assessed, preferably using a computer configured to automatically process video image (or other sensor) data to assess cranial and/or retinal perfusion. For example, if a compromised blood flow pattern is detected, different clinical options are available to attending physicians and anesthesiologists to correct the problem and prevent potential perioperative complications. Such clinical options include administration of thrombolytic, vasoactive, and/or cardiac inotropic drugs, administration of intravenous fluids or blood products, an increase in the output of cardiopulmonary bypass machine (if being used), tilting the operating room table, etc., which may allow for immediate and timely correction of the compromised blood flow.

As will be appreciated by those skilled in the art, the patient monitoring systems of the invention will provide precise information in real time to operating room personnel with respect to intracranial blood flow, which will allow for better clinical decisions to be made in those cases where intracranial hypoperfusion occurs. The instant systems will also provide assistance in anesthetic management of complex surgical cases.

In some embodiments, such monitoring allows trends in perfusion over time to be assessed. Such monitoring allows appropriate medical interventions to be undertaken in the event a state of hypoperfusion is detected, thereby reducing risks of brain hypoperfusion and stroke for patients undergoing heart surgery with cardiopulmonary bypass, as well as minimizing risk of retinal artery thrombosis with resulting blindness for patients undergoing prolonged procedures in the prone position.

In some preferred embodiments, continuous observation and monitoring of, for example, bilateral retinas in the operating room or other treatment areas allows real time comparison of left versus right retinal blood flow. This can be particularly useful for certain surgical procedures, such as carotid endarterectomy.

Kits

The invention also concerns kits that comprise one or more corneal caps of the invention (e.g., a pair of corneal caps) packaged in a sterile fashion in a suitable container, with or without instructions for use. After use, a corneal cap is typically properly discarded. Similarly, a kit containing an imaging system of the invention may be packaged in suitable container after manufacture.

Representative Embodiments

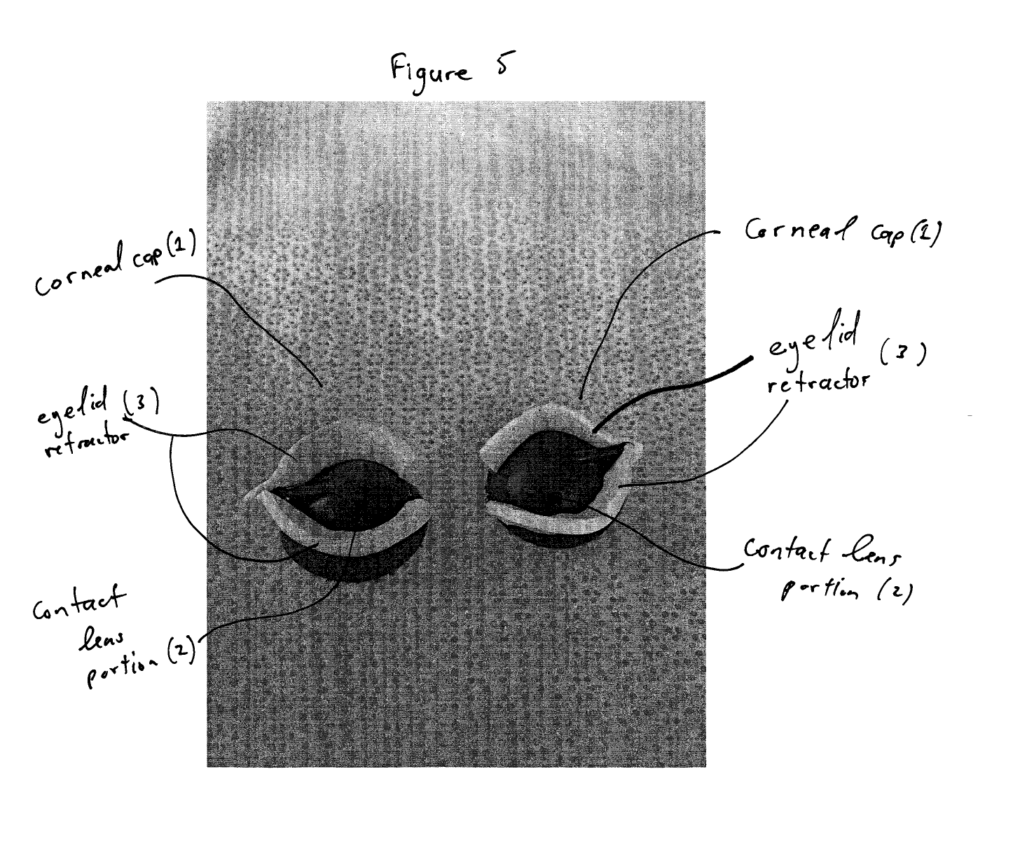

The following paragraphs describe several representative embodiments of the invention, with reference to FIGS. 5-8. In the figures, like elements are numbered alike.

FIG. 5 is an illustration that shows a pair of corneal caps (1). Each corneal cap includes a contact lens portion (2) and an eyelid retractor portion (3).

FIG. 6 shows a cross section of a representative corneal cap (1) positioned on the cornea (40) of a patient's eye (50). In this embodiment, the eyelid retractor portions (3) of the corneal cap (1) are outwardly tapered. In other embodiments, the eyelid retractor portion(s) (3) may not be tapered, or may even be tapered inwardly. As will be appreciated, the invention also envisions corneal cap embodiments wherein the eyelid retractor portion (3) and contact lens portion (2) are integrated or combined such that the eyelid retractor and contact lens portions are not readily distinguishable elements.

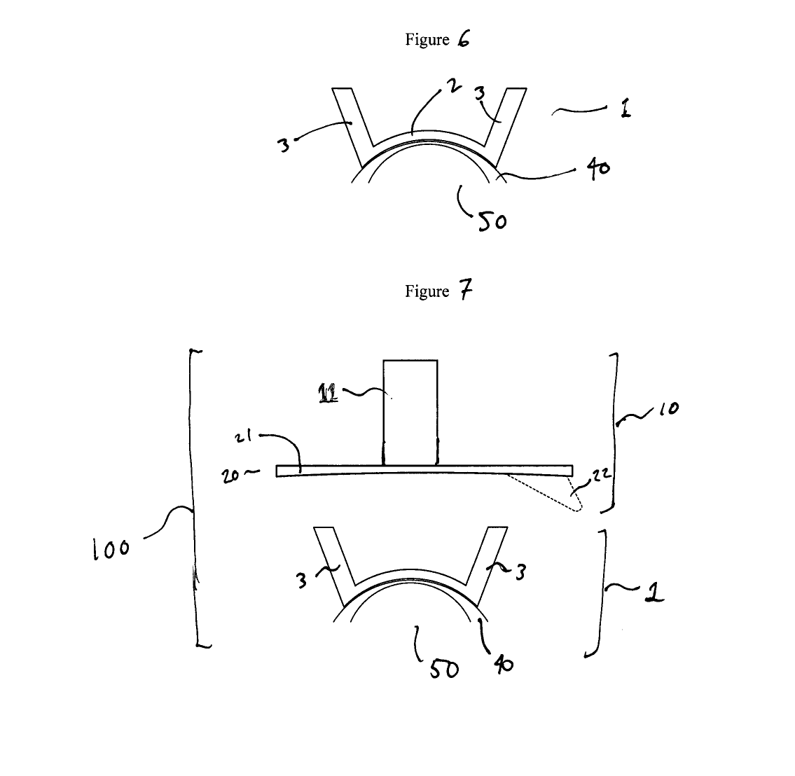

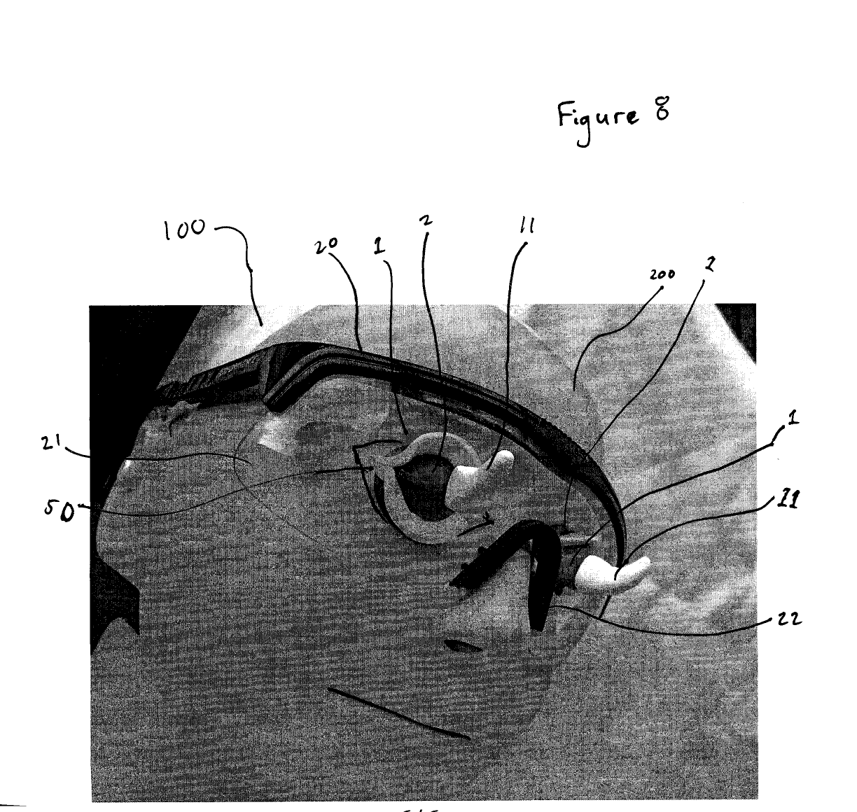

FIG. 7 illustrates a cross section of a patient monitoring system (100) positioned on a patient. Here, the corneal cap (1) is positioned on the cornea (40) of a patient's eye (50). The eyelid retractor portion (3) of the corneal cap prevents the patient's eyelids (not shown) from closing while the cap is positioned on the patient's eye (50). An imaging system (10) is shown positioned above the corneal cap (1) so that the retina and retinal vasculature of the patient's eye can be illuminated and imaged through the eye's pupil (not shown). The imaging system includes an image sensor, preferably a high definition video camera, light source, and mount (20). In the embodiment depicted here, the image sensor is a high definition video camera disposed in a housing (11) that also includes the light source, preferably LEDs. In this embodiment, the mount (20) is a pair of goggles having a lens (21) and a nose pad (22). The goggle frame is not shown. Note that in this embodiment, the corneal cap (1) does not contact the imaging system (10).

FIG. 8 also illustrates a patient monitoring system (100) positioned on a simulated patient (200). In this embodiment, the corneal cap (1) is positioned on the cornea of the patient's eye (50). The eyelid retractor portion (3) of the corneal cap prevents the patient's eyelids from closing while the cap is positioned on the patient's eye (50). An imaging system (10) is shown positioned above the corneal cap (1) so that the retina and retinal vasculature of the patient's eye can be illuminated and imaged through the eye's pupil (not shown). The imaging system (100) includes a high definition video camera, light source, and mount (20). In the embodiment depicted here, the high definition video camera is disposed in a housing (11) that also includes the light source, preferably LEDs. In this embodiment, the mount (20) is a pair of safety glasses having a transparent lens (21) and a nose pad (22).

The embodiments shown in the Figures and described above are representative only and not limiting.

This document describes in detail the inventive apparatus, methods, and articles of manufacture for patient monitoring. This was done for illustration purposes. Neither the specific embodiments of the invention as a whole, nor those of its features limit the general principles underlying the invention. The specific features described herein may be used in some embodiments, but not in others, without departure from the spirit and scope of the invention as set forth herein. Various physical arrangements of components and various step sequences also fall within the intended scope of the invention. Many additional modifications are intended in the foregoing disclosure, and it will be appreciated by those of ordinary skill in the art that in some instances some features of the invention will be employed in the absence of a corresponding use of other features. The illustrative examples therefore do not define the metes and bounds of the invention and the legal protection afforded the invention, which function is carried out by the claims and their equivalents.

I claim:

1. A corneal cap, comprising:
(a) a contact lens portion; and
(b) integrated on an outer surface of the contact lens portion, an eyelid retractor configured to prevent eyelid closure when the corneal cap is positioned on a patient's eye.

2. A corneal cap according to claim 1 wherein the contact lens portion is transparent.

3. A patient monitoring system, comprising:
(a) a corneal cap according to claim 1 for each eye of a patient that is to be monitored; and
(b) an imaging system configured to image retinal vasculature in the eye on which a corneal cap is positioned.

4. A patient monitoring system according to claim 3, wherein the imaging system comprises:
(a) for each eye to be monitored, an image sensor;
(b) a light source associated with each image sensor and positioned to illuminate the retina of the eye to be monitored;
(c) a mount configured to position the video camera(s) and light source(s) in front of a patient's eyes; and, optionally,
(d) a power supply.

5. A patient monitoring system according to claim 3, wherein each image sensor is included in a video camera, optionally a high definition video camera.

6. A patient monitoring system according to claim 3, wherein each light source is an LED light source.

7. A patient monitoring system according to claim 3, wherein the mount comprises goggles or an eyeglass frame adapted to removably secure the imaging system to the patient's head with the image sensor(s) and associated light source(s) positioned to image the retinal vasculature of the eye(s) to be monitored.

8. A patient monitoring system according to claim 3 that further comprises a pulse oximeter.

9. A patient monitoring system according to claim 3 that further comprises an intraocular pressure sensor.

10. A kit comprising a corneal cap according to claim 1.

11. A kit comprising a patient monitoring system according to claim 3.

12. A method of monitoring blood flow in a patient, comprising positioning a patient monitoring system according to claim 3 on a patient and monitoring blood flow in the retinal vasculature of at least one eye of the patient, thereby monitoring blood flow in a patient.

13. An automated method of continuous monitoring of retinal blood flow to assess in real time cerebral blood perfusion, comprising using a patient monitoring system according to claim 3 to monitor blood flow in the retinal vasculature of at least one eye of the patient and using a computer to process image data from the imaging system to generate a measured or derived index number reflecting a parameter associated with retinal blood flow, which index number is output to a user, and wherein a change in the index number reflects a change in the parameter.

14. An automated method according to claim 13 wherein the index number is indicative of a diameter of a retinal blood vessel, a number of pixels, optionally red pixels, in a selected area in one or more images captured by the imaging system, or a percentage change from a baseline.

15. A method of monitoring of bilateral retinas in real time, comprising using a patient monitoring system according to claim 3 to generate image data that allows for real time comparison of retinal blood flow in a patient's left eye versus the patient's right eye.
